SEQUENCE LISTING (2 sequences)

SEQ ID NO: 1 - DNA, 17 bp, synthetic construct
  misc_feature 1..17: note = primer 341F
  source 1..17: mol_type = other DNA
  variation 9: replace = g; replace = a; replace = c; replace = t
cctacgggnggcwgcag

SEQ ID NO: 2 - DNA, 21 bp, synthetic construct
  misc_feature 1..21: note = primer 785R
  source 1..21: mol_type = other DNA
gactachvgggtatctaakcc

(19) United States
(12) Patent Application Publication
(10) Pub. No.: US 2026/0069643 A1
(43) Pub. Date: Mar. 12, 2026

(54) COMBINATIONS COMPRISING VITAMIN B2 AND LACTOBACILLUS RHAMNOSUS

(71) Applicant: DSM IP Assets B.V., Maastricht (NL)

(72) Inventors: Thanh-Van PHAM, Kaiseraugst (CH); Wilbert SYBESMA, Kaiseraugst (CH); Robert STEINERT, Kaiseraugst (CH); Ateequr REHMAN, Kaiseraugst (CH)

(21) Appl. No.: 18/872,690

(22) PCT Filed: Jun. 8, 2023
(86) PCT No.: PCT/EP2023/065393

§ 371 Date: Dec. 6, 2024

(30) Foreign Application Priority Data
Jun. 10, 2022 (EP) 22178539.7

(51) Int. Cl. A61K 35/742; A61K 31/525; A61K 35/00; A61P 1/14

(52) U.S. Cl. CPC A61K 35/742; A61K 31/525; A61P 1/14; A61K 2035/115

(57) ABSTRACT

The present invention relates to a combination comprising vitamin B2 and Lactobacillus rhamnosus and its use for improving gut health in animals and humans. It was found that a combination of vitamin B2 and Lactobacillus rhamnosus, when delivered to the large intestine, increases the abundance of beneficial Verrucomicrobia, Akkermansiaceae and Akkermansia bacteria in the intestinal tract.

### CROSS-REFERENCE TO RELATED APPLICATIONS

This application is the U.S. national phase of International Application No. PCT/EP2023/065393 filed Jun. 8, 2023 which designated the U.S. and claims priority to EP 22178539.7 filed Jun. 10, 2022, the entire contents of each of which are hereby incorporated by reference.

### REFERENCE TO SEQUENCE LISTING SUBMITTED ELECTRONICALLY

The content of the electronically submitted sequence listing (Name: U.S. Pat. No. 18,872,690_SeqList_ST26.xml, Size: 4,450 bytes, and Date of Creation: Nov. 17, 2025) is herein incorporated by reference in its entirety.

### FIELD OF THE INVENTION

The present invention relates to combinations comprising vitamin B2 and Lactobacillus rhamnosus and uses thereof in improving gut health in animals and humans. It was found that a combination of vitamin B2 and Lactobacillus rhamnosus, when delivered to the large intestine, increases the abundance of specific beneficial bacteria in the intestinal tract.

### BACKGROUND OF THE INVENTION

Increasing evidence indicates that imbalances in the human gut microbiota (also referred to as “dysbiosis”) may be associated with Western diseases, including obesity and type 2 diabetes, as well as cardiovascular, autoimmune, and intestinal inflammatory disease. Thus, targeted modulation of the human gut microbiome intended to restore imbalances represents a potential therapeutic and preventive strategy and has attracted the attention of academics as well as those engaged in various industries. Public awareness and acceptance of substances that modulate the human gut microbiome continue to grow.

There is a consensus that certain live microorganisms have beneficial effects on human health. Verrucomicrobia are mucin-degrading bacteria residing in the intestinal mucosa that contribute to intestinal health and glucose homeostasis, and play as an interface between the human gut microbiome and host tissues. Higher proportions of Verrucomicrobia are associated with better sleep quality and cognitive performance in adults (Anderson, J. R. et al. A preliminary examination of gut microbiota, sleep, and cognitive flexibility in healthy older adults (2017).

Akkermansiaceae is the dominant family in the phylum Verrucomicrobia. The abundance of Akkermansiaceae was found to be significantly increased in prostate cancer-bearing mice, implying a role in prostate cancer (Pin-Yu H. et al., Int. J Molecular Sciences, 2021:9626).

Akkermansia belong to the bacterial family of Akkermansiaceae and produce butyrate and propionate. Akkermansia have been shown to have an anti-obesity protective effect in previous studies and may be used as promising probiotics (Zhou Q et al, Gut bacteria Akkermansia is associated with reduced risk of obesity: evidence from the American Gut Project 2020).

Among commensal bacteria residing in the intestine, Akkermansia muciniphila has attracted growing interest for its health-promoting effects. Akkermansia muciniphila is highly abundant in lean and non-diabetic individuals, and is linked to lower rates of obesity and associated metabolic disorders (Dao MC. Akkermansia muciniphila and improved metabolic health during a dietary intervention in obesity: relationship with gut microbiome richness and ecology (2016)). In contrast, several studies reported a reduction of Akkermansia muciniphila levels in various disorders and diseases in humans, including intestinal diseases, such as inflammatory bowel disease, as well as extra-intestinal diseases, such as autism, atopy or obesity and related diseases. Akkermansia muciniphila has also been found to be lower in several conditions such as diabetes, intestinal inflammation, liver diseases, or chronic alcohol consumption. This is associated with an altered gut barrier function which eventually triggers low-grade inflammation and metabolic disorders. The presence of Akkermansia muciniphila is associated with a healthier status in humans. Indeed, numerous studies have found that Akkermansia muciniphila is positively associated with a healthy gut lining, reduced metabolic disorders and decreased low-grade inflammation.

In the context of obesity-associated disorders, Akkermansia muciniphila is systematically found to be inversely correlated with cardiometabolic risk factors. These factors include insulin resistance, serum lipids, BMI and adiposity. In contrast, it is positively correlated with protective markers such as high-density lipoprotein (HDL, or “good” cholesterol).

Akkermansia muciniphila acts as a guardian of the intestinal barrier function through its unique mucus-degrading abilities and its location within the mucus layer. It stimulates mucin production through mucin degradation, renewing the mucus layer. In a healthy human gut, these bacteria maintain mucus production and thickness, thus preventing the invasion of pathogenic bacteria (Zhai Q. et al., A next generation probiotic, Akkermansia muciniphila (2019) and Derrien M. et al. Akkermansia muciniphila and its role in regulating host functions (2017)).

Recently, it was demonstrated that vitamins may modulate the human gut microbiome. WO2020/043797 discloses that vitamins can be useful to increase the growth of certain beneficial bacteria in the intestine. However, WO2020/043797 does not describe that vitamins can be used in combination with probiotics to increase the abundance of other beneficial bacteria. Moreover, the human gut is home to hundreds of different microbes, and it would be desirable to be able to boost specific beneficial bacteria. Specifically, it would be desirable to increase the abundance of Verrucomicrobia, Akkermansiaceae, and Akkermansia bacteria in the intestine to enhance wellness, improve health, and support the immune system.

### SUMMARY OF THE INVENTION

The present invention relates to the following items:
1) Combination comprising vitamin B2 and Lactobacillus (Lacticaseibacillus) rhamnosus.
2) Combination according to item 1, wherein said combination comprises vitamin B2 and a Lactobacillus rhamnosus GG, preferably Lactobacillus rhamnosus DSM 32550.
3) Combination according to item 1 or item 2, wherein said combination is for simultaneous administration or delivery or consumption, and preferably wherein said combination is a fixed combination.
4) Combination according to item 1 or item 2, wherein said combination is for sequential administration or delivery or consumption, and preferably wherein said combination is a free combination.
5) Combination according to any one of item 1-4, wherein said combination is an oral dosage form, and wherein said combination is more preferably a solid oral dosage form.
6) Combination according to any one of items 1-5, wherein said combination is for administration or delivery to the large intestine.
7) Combination according to any one of item 1-6 for use as a medicament, a dietary supplement, or a food supplement.
8) Combination according to any one of item 1-7 for use in the treatment of a patient that is in need of increasing the abundance of Verrucomicrobia, Akkermansiaceae, and/or Akkermansia in the large intestine.
9) Combination for the use according to item 8, wherein said patient is in need of increasing the abundance of Akkermansiaceae and is suffering from prostate cancer.
10) Combination according to any one of item 1-7 for use in the treatment of a patient that is in need of increasing the abundance of Akkermansia muciniphila in the large intestine.
11) Combination for the use according to item 10, wherein said patient is suffering from one or more of the following conditions: obesity, diabetes, intestinal inflammation, liver disease, autism, atopy, metabolic disorder, and chronic alcohol consumption.
12) Combination comprising vitamin B2 and Lactobacillus (Lacticaseibacillus) rhamnosus for use in increasing the abundance of Verrucomicrobia, Akkermansiaceae, and/or Akkermansia in the large intestine of an animal, preferably a human, wherein said use comprises delivering the vitamin B2 and the Lactobacillus rhamnosus to the large intestine.
13) Combination comprising vitamin B2 and Lactobacillus rhamnosus for the use according to item 12, wherein the vitamin B2 and the Lactobacillus rhamnosus are delivered to the large intestine by a delayed-release formulation.
14) Combination comprising vitamin B2 and Lactobacillus rhamnosus for the use according to item 12 or item 13, wherein said use comprises administering the vitamin B2 and the Lactobacillus rhamnosus simultaneously and/or sequentially to the animal, preferably a human.
15) Combination comprising vitamin B2 and Lactobacillus rhamnosus for the use according to any one of item 12-14, wherein the animal, including a human, is experiencing at least one condition selected from: prostate cancer, obesity, diabetes, intestinal inflammation, liver disease, autism, atopy, metabolic disorder, and chronic alcohol consumption.
16) Combination comprising vitamin B2 and Lactobacillus rhamnosus for the use according to any one of item 12-15, wherein the Lactobacillus rhamnosus is a Lactobacillus rhamnosus GG, preferably Lactobacillus rhamnosus DSM 32550.

### BRIEF DESCRIPTION OF THE DRAWINGS

FIG. 1: Relative abundance (%) of bacteria of the phylum Verrucomicrobia in the mucus of the transverse colon upon treatment with Lactobacillus rhamnosus GG in the presence or absence of vitamin B2, at the end of the control period (n=3) and at the end of the treatment period (n=3). Statistically significant differences are indicated with ‘*’ (p<0.05).

FIG. 2: Relative abundance (%) of bacteria of the family Akkermansiaceae in the mucus of the transverse colon upon treatment with Lactobacillus rhamnosus GG in the presence or absence of vitamin B2, at the end of the control period (n=3) and at the end of the treatment period (n=3). Statistically significant differences are indicated with ‘*’ (p<0.05).

FIG. 3: Relative abundance (%) of species Akkermansia muciniphila in the mucus of the transverse colon upon treatment with Lactobacillus rhamnosus GG in the presence or absence of vitamin B2, at the end of the control period (n=3) and at the end of the treatment period (n=3). Statistically significant differences are indicated with ‘*’ (p<0.05).

### DETAILED DESCRIPTION OF THE INVENTION

Verrucomicrobia, Akkermansiaceae, and Akkermansia are bacteria known for their beneficial effects on human health. The present inventors have found that vitamin B2 in combination with Lactobacillus rhamnosus can boost the growth of Verrucomicrobia, Akkermansiaceae, and Akkermansia muciniphila bacteria in the large intestine, leading to an increase of Verrucomicrobia, Akkermansiaceae and Akkermansia muciniphila levels in the gut.

Hence, in a first aspect, the present invention relates to combinations comprising vitamin B2 and Lactobacillus (Lacticaseibacillus) rhamnosus. Preferably, the Lactobacillus (Lacticaseibacillus) rhamnosus is a Lactobacillus rhamnosus GG strain, more preferably it is Lactobacillus rhamnosus DSM 32550. The combination is for simultaneous and/or sequential administration.

Patent claims relating to a “combination” are product claims. The product of the present invention comprises two active ingredients: a vitamin (vitamin B2) and a probiotic (Lactobacillus rhamnosus). For simultaneous and/or sequential administration, see definitions and embodiments below.

Vitamin B2 (also known as riboflavin) is one of the water-soluble B vitamins which is an essential component of two major coenzymes, flavin mononucleotide (FMN; also known as riboflavin-5′-phosphate) and flavin adenine dinucleotide (FAD). These coenzymes play major roles in energy production; cellular function, growth, and development; and metabolism of fats, drugs, and steroids. Riboflavin can be purchased from DSM GmbH. Alternative suppliers are, for example, TER Chemicals Distribution Group, BIOCHEM Bernburg GmbH, DVA International GmbH, Falken Trade GmbH, and Neupert Ingredients GmbH.

The most common Lactobacillus (Lacticaseibacillus) rhamnosus strain is Lactobacillus rhamnosus GG. It can be purchased, for example, from Chr. Hansen, Denmark, as LGG®. Lactobacillus (Lacticaseibacillus) rhamnosus DSM 32550 (Biocare Copenhagen, Denmark) has a genomic sequence which is 99.99% identical to the genomic sequence of LGG®. It can thus be considered that L. rhamnosus DSM 32550 is identical or equivalent to LGG® for practical purposes. Therefore, L. rhamnosus DSM 32550 will herein be referred to as a “Lactobacillus rhamnosus GG”.

Alternative Lactobacillus rhamnosus strains are, inter alia, Lactobacillus rhamnosus HN001 (Howaru; Danisco/DuPont), Lactobacillus rhamnosus GR-1® (Chr. Hansen, Denmark), and Lactobacillus rhamnosus Rosell-11 (Lallemand, Canada).

Lactobacillus (Lacticaseibacillus) rhamnosus DSM 32550 (Biocare Copenhagen) is a preferred strain according to the present invention. It has been deposited at Leibniz Institute DSMZ-German Collection of Microorganisms and Cell Cultures, Inhoffenstr. 7B, D-38124 Braunschweig, Germany, according to the Budapest Treaty on 6. July 2017. The accession number given by the International Depository Authority is DSM 32550.

In one embodiment, the combination of the present invention is for simultaneous administration. Preferably, the combination for simultaneous administration is a fixed combination. However, for simultaneous administration, a free combination can also be used.

In another embodiment, the combination is for sequential administration. The combination for sequential administration is a free combination.

Preferably, the combination is an oral dosage form; more preferably, it is a solid oral dosage form.

The combination of the present invention can be, for example, a pharmaceutical combination or composition, a dietary supplement, or a food supplement.

In another aspect, the present invention relates to vitamin B2 and Lactobacillus rhamnosus (i.e., a combination of vitamin B2 and Lactobacillus rhamnosus) for use as a medicament.

Preferably, the combinations of the invention (e.g. pharmaceutical combinations) are for use in the treatment of a patient that is in need of increasing the abundance of Verrucomicrobia, Akkermansiaceae, and/or Akkermansia in the large intestine. In one embodiment, said patient is in need of increasing the abundance of Akkermansiaceae and is suffering from prostate cancer.

In another preferred embodiment, the (pharmaceutical) combinations of the invention are for use in the treatment of a patient that is in need of increasing the abundance of Akkermansia muciniphila in the large intestine. Preferably, said patient is suffering from at least one condition selected from: obesity, diabetes, intestinal inflammation, liver disease, autism, atopy, metabolic disorder, and chronic alcohol consumption.

In a further aspect, the present invention relates to vitamin B2 and Lactobacillus rhamnosus (i.e., a combination of vitamin B2 and Lactobacillus rhamnosus) for use in improving gut health in an animal. Said improvement comprises or consists of increasing the abundance of Verrucomicrobia, Akkermansiaceae, and/or Akkermansia in the large intestine of said animal. Specifically, the vitamin B2 and Lactobacillus rhamnosus is for use in increasing the abundance of Verrucomicrobia, Akkermansiaceae, and/or Akkermansia in the large intestine (colon) of an animal, wherein said use preferably comprises delivering the vitamin B2 and Lactobacillus rhamnosus to the large intestine. Preferably, the animal is a human.

To achieve an increase of the abundance of Verrucomicrobia, Akkermansiaceae, and/or Akkermansia in the large intestine, the vitamin B2 and Lactobacillus rhamnosus is preferably directly delivered to the large intestine. That is, the vitamin is delivered/administered in a manner such that the vitamin is not absorbed in the stomach and/or small intestine; rather the vitamin and the probiotic is delivered/administered to the distal intestinal tract, preferably the large intestine (colon). This is preferably done by delivering/administering the vitamin B2 and Lactobacillus rhamnosus in a delayed-release formulation. Oral administration is preferred.

In a preferred embodiment, the Akkermansia bacteria to be boosted are of the species Akkermansia muciniphila and the animal (including a human) is experiencing one or more condition(s) selected from the group consisting of: obesity, diabetes, intestinal inflammation, liver disease, autism, atopy, metabolic disorder, and chronic alcohol consumption.

Preferably, the Lactobacillus rhamnosus used is a Lactobacillus rhamnosus GG. Lactobacillus rhamnosus DSM 32550 is particularly preferred.

In another aspect, the present invention relates to a method of increasing the abundance of Verrucomicrobia, Akkermansiaceae, and/or Akkermansia in the intestine, preferably the large intestine, comprising administering to the animal an effective dose of vitamin B2 and Lactobacillus rhamnosus (preferably, Lactobacillus rhamnosus GG; in particular Lactobacillus rhamnosus DSM 32550). The method is for improving intestinal health in an animal, including a human, wherein said improvement comprises increasing the abundance of Verrucomicrobia, Akkermansiaceae, and/or Akkermansia in the large intestine. Preferably, the animal is a human. Preferably, the vitamin B2 and the Lactobacillus rhamnosus is delivered directly to the large intestine. Delivery to the large intestine can be achieved by administering the vitamin B2 and the Lactobacillus rhamnosus as a delayed-release formulation.

The methods of the invention can be used to treat, prevent, and/or lessen the symptoms in an animal, including a human, in need thereof, of one or more of the following: prostate cancer; obesity, diabetes, intestinal inflammation, liver disease, autism, atopy, metabolic disorder, and chronic alcohol consumption.

In a further aspect, the present invention relates to the use of vitamin B2 and Lactobacillus rhamnosus for increasing the abundance of Verrucomicrobia, Akkermansiaceae, and/or Akkermansia (preferably, Akkermansia muciniphila) in the large intestine of an animal, preferably a human, wherein said use comprises delivering the vitamin B2 and the Lactobacillus rhamnosus to the large intestine. Preferably, the use comprises delivering/administering the vitamin B2 and Lactobacillus rhamnosus to the large intestine by a delayed-release formulation. Preferably, the animal, including a human, is experiencing one or more condition(s) selected from the group consisting of: prostate cancer; obesity, diabetes, intestinal inflammation, liver disease, autism, atopy, metabolic disorder, and chronic alcohol consumption.

In the combinations, uses, and methods of the invention, preferably, the vitamin B2 dose is up to 200 mg/day, preferably 5-100 mg/day, more preferably from 10-50 mg/day. In one embodiment, vitamin B2 is dosed/administered in an amount such that its local concentration in the colon is at least 0.001 g/L, preferably at least 0.01 g/L more preferably at 0.02 g/L. Preferred local concentrations in the colon range from about 0.001 g/L to about 0.5 g/L or from about 0.005 g/L to about 0.2 g/L, preferably about 0.01 to about 0.02 g/L.

The dosage of the Lactobacillus rhamnosus can be up to 5E+10 cfu/day. Preferably, the dosage range is from 1E+08 to 1E+10 cfu/day, more preferably from 1E+09 to 5E+10 cfu/day. Preferably, the Lactobacillus rhamnosus is a Lactobacillus rhamnosus GG. Lactobacillus rhamnosus DSM 32550 is particularly preferred.

### Definitions and Embodiments

As used throughout, the following definitions apply:

Patent claims relating to a “combination” or “pharmaceutical combination” are product claims. The product of the present invention comprises two active ingredients: a vitamin (vitamin B2) and a probiotic (Lactobacillus rhamnosus).

A “combination for simultaneous administration” or a “combination for simultaneous consumption” is a combination that is suitable for simultaneous administration or consumption, respectively. By “simultaneous administration” or “simultaneous consumption”, it is meant that the vitamin and the probiotic bacteria are administered/consumed on the same day (i.e., with 24 hours). Said two active ingredients can be administered/consumed at the same time (for fixed combinations) or one at a time (for free combinations). For example, the vitamin can be administered/consumed in one pill or tablet, while the probiotic is administered/consumed in another pill or tablet, wherein both pills/tablets are administered/consumed within 24 hours. In another example, the vitamin and the probiotic are formulated in the same composition and are administered/consumed at exactly the same time.

A “combination for sequential administration or consumption” is a combination that is suitable for sequential administration or consumption, respectively. By “sequential administration” or “sequential consumption”, it is meant that during a period of two or more days of continuous treatment, only one of the vitamin and the probiotic is administered/consumed on any given day. By way of example, the vitamin can be administered/consumed on day one, and the probiotic is administered/consumed only the next day (i.e., after more than 24 hours), or even later. The active ingredients can be administered/consumed in any order.

A “fixed combination” is a combination that delivers both actives (i.e., the vitamin and the probiotic) at the same time to a patient. A solid oral dosage form (e.g., a tablet or capsule) comprising both, the vitamin and the probiotic, is an example of a fixed combination. A liquid oral dosage form (e.g., oral drops) comprising both, the vitamin and the probiotic, is another example of a fixed combination.

A “free combination” is a combination that allows to administer/consume both actives (i.e., the vitamin and the probiotic) separately, i.e. one at a time. Treatment regimens in which the vitamin and the probiotic are not administered/consumed by the same route and/or are not administered/consumed at the same time require free combinations.

Simultaneous administration/consumption can be done both by using a fixed combination and a free combination. Sequential administration/consumption requires a free combination; fixed combinations are not suitable for sequential administration/consumption. Hence, free combinations are more versatile: they are suitable for sequential administration/consumption and—if both actives are administered/consumed on the same day—also for simultaneous administration/consumption. Fixed combinations are only suitable for simultaneous administration/consumption if both ingredients (i.e., the vitamin and the probiotic) are to be administered/consumed at the same time of the same day; if, however, the vitamin and the probiotic are to be administered/consumed on the same day but separately, fixed combinations are not suitable.

By “separate administration/consumption”, it is meant that the vitamin and the probiotic are administered one at a time. Thus, separate administration/consumption can refer to both sequential administration/consumption and—when referring to the administration/consumption of both actives on the same day but one at a time—also to simultaneous administration/consumption.

“Administering” or “administration” means to give or to deliver an active to a human or animal; likewise, the human or animal can take (consume) the active.

The term “vitamin B2”, which is used interchangeably with “riboflavin”, includes riboflavin and esters thereof, in particular riboflavin-5′-phosphate and other pharmaceutically acceptable forms.

To “increase the abundance” of Verrucomicrobia, Akkermansiaceae, or Akkermansia means to increase the level (or the amount, or number, or the population size) of Verrucomicrobia, Akkermansiaceae, or Akkermansia compared to the respective control (i.e., the level/amount/number/population size of Verrucomicrobia, Akkermansiaceae, or Akkermansia when the combination of vitamin B2 and Lactobacillus rhamnosus has not been added).

The term “intestine” (or “gut”) as used herein refers to the portion of the gastrointestinal tract consisting of the small intestine and the large intestine. The “large intestine” (Intestinum crassum) is the lower part of the gastrointestinal tract and is also referred to herein as “colon”.

“Direct delivery” or “directly delivered” means that the vitamin is formulated in a manner such that the vitamin is not absorbed in the stomach and/or small intestine; rather the vitamin is made available in the distal intestinal tract, preferably the large intestine (colon), where it is available to the microbiome. The vitamin is not part of a person's usual daily nutritional requirements (generally obtained through diet and conventional vitamin supplementation), and is administered in excess thereof. For human use, the preferred method according to the present invention is through a form which delays release until the large intestinal tract (colon) is reached. Alternatively, a large enough dose can be administered, so that only a portion of the administered vitamin is absorbed in the proximal small intestine, and the remainder, which is an effective dose, is available to the large intestinal tract; although not preferred, the latter method of delivery can be used for humans as well. With respect to the probiotic, “direct delivery” or “directly delivered” means that the probiotic is formulated in a manner such that it is not released in the stomach and/or small intestine but rather it is made available in the distal intestinal tract, preferably the large intestine (colon).

A used herein, “delayed release” refers to the release of the vitamin and/or the probiotic at a time later than immediately after administration. Preferably, “delayed release” means delivery of the vitamin (and/or probiotic), upon oral administration, to the large intestine (colon) in a delayed manner relative to an immediate release formulation.

An “enteric layer” or “enteric coating” is a layer surrounding a core, wherein the core comprises the active agent and the layer confers resistance to gastric juice.

“Prevent” can include lessening the risk of an adverse condition occurring, lessening the symptoms of an adverse condition, lessening the severity of an adverse condition, and prolonging the time for occurrence of an adverse condition.

“Oral formulation” means that the vitamin and/or probiotic is formulated for oral administration/consumption.

“Co-administering” or “co-administration” means that the vitamin and/or the probiotic is delivered/administered/consumed simultaneously (i.e., together), or separately but within a time frame of 24 hours. The vitamin can be delivered/administered/consumed first. Likewise, the probiotic can be delivered/administered/consumed first.

“Lactobacillus rhamnosus” was recently renamed “Lacticaseibacillus rhamnosus”; both names are used interchangeably herein, and both can be abbreviated as “L. rhamnosus”.

### Doses

Preferably, vitamin B2 can be administered in an amount such that its local concentration in the colon is at least 0.001 g/L, preferably at least 0.01 g/L more preferably at 0.02 g/L. Preferred local concentrations in the colon range from about 0.001 g/L to about 0.5 g/L or from about 0.005 g/L to about 0.2 g/L, preferably about 0.01 to about 0.02 g/L. Specific dosages per day can range up to 200 mg/day, preferably 5-100 mg/day, more preferably from 10-50 mg/day.

The dosage of the probiotic can be up to 5E+10 cfu/day. Preferably, the dosage range of the probiotic is from 1E+08 to 1E+10 cfu/day, more preferably from 1E+09 to 5E+10 cfu/day.

### Formulations

The vitamin (vitamin B2) and/or the probiotic (Lactobacillus rhamnosus), preferably both, is (are) preferably present in a formulation which allows the vitamin (and/or probiotic) to be available predominantly in the large intestine.

Oral formulations are preferred. Other formulations include non-oral routes, such as via suppositories or injections.

For human use, the preferred method is through a delayed-release form which delays delivery until the intestinal tract is reached. For non-human animals, a preferred delivery includes a method of administering a large enough dose so that only a portion of the vitamin and/or probiotic delivered is absorbed in the stomach, and the remainder, which is an effective dose, is available to the intestinal tract; although not preferred, this method of delivery can be used for humans as well.

Delayed-release formulations are known in the art. Preferably, the delayed-release formulations have an enteric coating (also referred to as enteric layer).

In one embodiment of the present invention, the vitamin and/or probiotic, preferably both, is in a formulation comprising an enteric capsule, filled with a composition comprising the vitamin and/or probiotic. The enteric capsule confers resistance against the acidic environment of the stomach. For example, soft gel formulations may deliver the active agent in solution and yet offer advantages of solid dosage forms.

In another embodiment, the formulation is a tablet comprising (i) a core comprising the vitamin and/or the probiotic, and (ii) a delayed-release coating such as an enteric coating. This may be a hard gel capsule.

Alternatively, for direct colon delivery, a matrix-based delivery system can be used. Matrix based systems have no discrete layer of coating material, but the active agent (i.e., the vitamin and/or the probiotic) is more or less homogenously distributed within the matrix. Further, there are colon-release systems that embed the active agent in e.g. in a fiber matrix (enzyme-triggered) and an enteric coating on top.

The release of the vitamin and/or probiotic may be delayed until the small intestine. In another embodiment, the release is delayed until the distal small intestine. In yet another, preferred embodiment, the release of the vitamin and/or probiotic is delayed until the colon (large intestine).

In a preferred embodiment for humans, the vitamin and/or probiotic is formulated in a solid dosage form for oral administration. The formulation may be in the form of a capsule, pellet, bead, sphere, mini spheres, tablet, mini tablet, or granule, optionally coated with a delayed release coating that prevents the release of the active agent before the small intestine, preferably before the colon.

Coating, or matrix materials for the delayed release of the vitamin and/or probiotic, in particular for targeted release in the ileum or the large intestine upon oral administration are known in the art. They can be subdivided into coating materials that disintegrate above a specific pH, coating materials that disintegrate after a specific residence time in the gastrointestinal tract and coating materials that disintegrate due enzymatic triggers specific to the microflora of a specific region of the intestines. Coating materials from different categories are commonly used in combinations.

Coating materials of the different categories for targeting to the large intestine have been reviewed for example in Bansal et al. (Polim. Med. 2014, 44, 2,109-118). In one embodiment of the present invention, the delayed-release coating comprises at least one component selected from coating materials that disintegrate pH-dependently, coating materials that disintegrate time-dependently, coating materials that disintegrate due to enzymatic triggers in the intestinal environment (e.g., in the intestinal environment of the ileum and the large intestine), and combinations thereof.

Coating materials that disintegrate pH-dependently include polyvinyl acetate phthalate, cellulose acetate trimellitate, hydroxypropyl methylcellulose phthalate HP-50, HP-55 or HP-55S, cellulose acetate phthalate, shellac, hydroxypropyl methylcellulose acetate succinate (HPMCAS), poly(methacrylic acid, ethyl acrylate) 1:1 (Eudragit® L100-55, Eudragit® L30D-55), poly(methacrylic acid, methyl methacrylate) 1:1 (Eudragit®) L-100, Eudragit® L12.5), poly(methacrylic acid, methyl methacrylate) 1:2 (Eudragit® S-100, Eudragit® S12,5, and Eudragit® FS30D). Coating materials that disintegrate time-dependently include Eudragit® RL, Eudragit®RS, and ethylcellulose. Coating materials that disintegrate due to enzymatic triggers in the large intestinal environment include chondroitin sulfate, pectin, guar gum, chitosan, inulin, lactulose, raffinose, stachyose, alginate, dextran, xanthan gum, locust bean gum, arabinogalactan, cyclodextrin, pullulan, carrageenan, scleroglucan, chitin, curdulan, levan, amylopectin, starch, amylose, resistant starch, and azo compounds being degraded by azo bonds splitting bacteria.

The following non-limiting examples are presented to better illustrate the invention.

### EXAMPLES

The aim of this study was to investigate the effect of a combination of vitamin B2 and Lactobacillus rhamnosus on the composition of the gut microbiota in a long-term continuous fermentation experiment.

### Materials and Methods

### Design of the Long-Term SHIME Fermentation Experiment (Colon Model)

The typical reactor setup of the SHIME®, representing the gastrointestinal tract of the adult human, was described by Molly et al. (1993) Applied Microbiology and Biotechnology 39(2):254-258. Inoculum preparation, retention time, pH, temperature settings and reactor feed composition were previously described by Possemiers et al. (2004) FEMS Microbiol Ecol. 49(3):495-507. Compared to the typical setup of the SHIME, the long-term SHIME experiment used for this example included some adaptations. In one reactor, at first, the conditions of the stomach were simulated, before being changed by the computer to conditions simulating the small intestine. The suspension was then added to the colonic reactors mimicking the transverse colon (pH 6.15-6.4; retention time=32 h; volume of 800 mL).

The SHIME® experiment for this study consisted of three stages:
1. Stabilization period: After inoculation of the colon reactors with an appropriate fecal sample, a two-week stabilization period allowed the microbial community to differentiate in the different reactors depending on the local environmental conditions. During this period the basic nutritional matrix was provided to the SHIME to support the maximum diversity of the gut microbiota originally present in the faecal inoculum.
2. Control period: During this two-week reference period, the standard SHIME nutrient matrix was further dosed to the model for a period of 14 days. Analysis of samples in this period allowed to determine the baseline microbial community composition and activity in the different reactors, which is used as a reference for results obtained during the treatment.
3. Treatment: During this three-week period, the SHIME was operated under nominal conditions, but the appropriate probiotic strain and vitamin(s) were supplemented to the appropriate reactors. The probiotic strain was added to the reactors at a concentration of 1*10¹⁰ CFU/reactor. Vitamin B2 (Riboflavin, DSM) was added to the reactors at a dose of 10 mg/day.

The probiotic strain used in this experiment was the Lactobacillus rhamnosus GG equivalent Lactobacillus rhamnosus DSM 32550 (Biocare Copenhagen).

Lactobacillus rhamnosus DSM 32550 has a genomic sequence which is 99.99% identical to the genomic sequence of LGG®. It can therefore be considered that L. rhamnosus DSM 32550 is identical or equivalent to LGG® for practical purposes. In the Examples and Figures herein, Lactobacillus rhamnosus DSM 32550 will thus be referred to as a Lactobacillus rhamnosus GG strain.

Quantitative Microbial Community Analysis by 16S rRNA Gene Sequencing and Flow Cytometry

Samples for quantitative 16S-targeted Illumina sequencing were collected 3×/week during the last week of the control and treatment period. Next-generation 16S rRNA gene amplicon sequencing of the V3-V4 region was performed by LGC Genomics GmbH (Berlin, Germany) on samples from the medium-term SHIME experiment. Library preparation and sequencing were performed on an Illumina MiSeq platform with v3 chemistry. The 341F (50-CCTACGGGNGGCWGCAG-30) and 785R (50-GACTACHVGGGTATCTAAKCC-30) primers were used as described by De Paepe et al. (2017) with the reverse primer being adapted to increase coverage. Quality control PCR was conducted using Taq DNA Polymerase with the Fermentas PCR Kit according to the manufacturers' instructions (Thermo Fisher Scientific, Waltham, MA, USA). The DNA quality was verified by electrophoresis on a 2% (w/v) agarose gel for 30 min at 100 V. Bioinformatics analysis of amplicon data was performed as described by De Paepe et al. (2017). The obtained high-resolution proportional phylogenetic information (i.e., proportional abundances (%)) was combined with an accurate quantification of total bacterial cells via flow cytometry to obtain quantitative data at phylum, family, and species level. This was done by multiplying the proportional abundances with absolute cell numbers (cells/mL) obtained via flow cytometry. For flow cytometry analysis, 10-fold serial dilutions were prepared in Dulbecco's phosphate-buffered Saline (DPBS) (Sigma-Aldrich, Bornem, Belgium) of all samples and stained with 0.01 mM SYTO24 (Life Technologies Europe, Merelbeke, Belgium) for 15′ at 37° C. in the dark. Samples were analyzed on a BD Facsverse (BDBiosciences, Erembodegem, Belgium) using the high-flowrate setting and bacteria were separated from medium debris and signal noise by applying a threshold level of 200 on the SYTO channel.

### Results

Supplementation of a Combination of Lactobacillus rhamnosus GG and Vitamin B2 Increased the Abundance of Verrucomicrobia

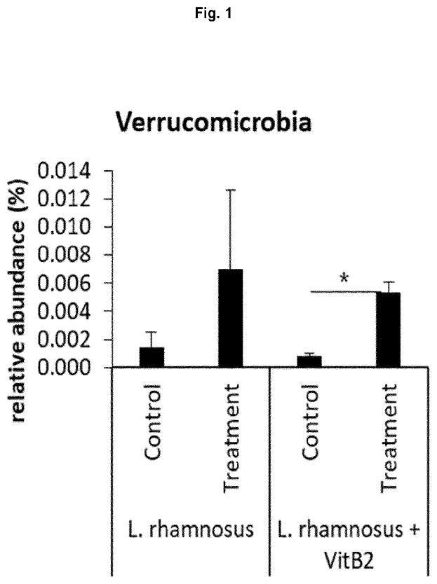

As can be taken from FIG. 1, supplementation of Lactobacillus rhamnosus GG alone did not significantly change the abundance of Verrucomicrobia compared to the control. In contrast, the combination of Lactobacillus rhamnosus GG and vitamin B2 significantly increased the abundance of Verrucomicrobia compared to the control.

Supplementation of a Combination of Lactobacillus rhamnosus GG and Vitamin B2 Increased the Abundance of Akkermansiaceae

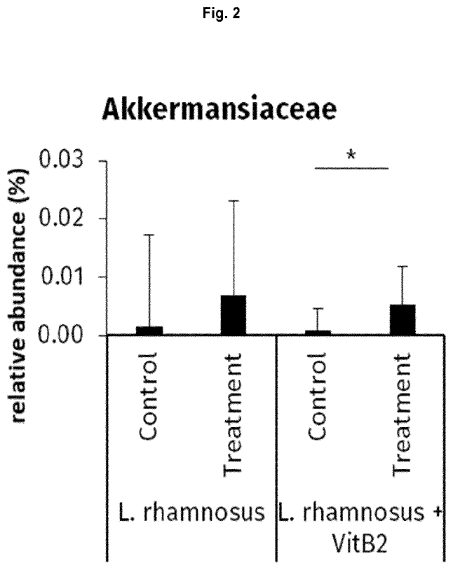

As can be taken from FIG. 2, supplementation of Lactobacillus rhamnosus GG alone did not significantly change the abundance of Akkermansiaceae compared to the control. In contrast, the combination of Lactobacillus rhamnosus GG and vitamin B2 significantly increased the abundance of Akkermansiaceae compared to the control.

Supplementation of a Combination of Lactobacillus rhamnosus GG and Vitamin B2 Increased the Abundance of Akkermansia muciniphila

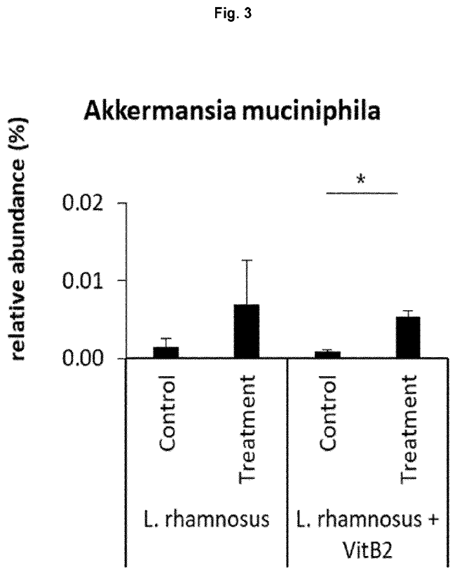

As can be taken from FIG. 3, supplementation of Lactobacillus rhamnosus GG alone did not significantly change the abundance of Akkermansia muciniphila compared to the control. In contrast, the combination of Lactobacillus rhamnosus GG and vitamin B2 significantly increased the abundance of Akkermansia muciniphila compared to the control.

1. Combination comprising vitamin B2 and Lactobacillus rhamnosus.

2. Combination according to claim 1, wherein said combination comprises vitamin B2 and Lactobacillus rhamnosus GG.

3. Combination according to claim 1, wherein said combination is for simultaneous administration or consumption.

4. Combination according to claim 1, wherein said combination is for sequential administration or consumption.

5. Combination according to claim 1, wherein said combination is an oral dosage form, and wherein said combination is more preferably a solid oral dosage form.

6. Combination according to claim 1, wherein said combination is for administration to the large intestine.

7. Combination according to claim 1 for use as a medicament, a dietary supplement, or a food supplement.

8. Combination according to claim 1 for use in the treatment of a patient that is in need of increasing the abundance of Verrucomicrobia, Akkermansiaceae, and/or Akkermansia in the large intestine.

9. Combination for the use according to claim 8, wherein said patient is in need of increasing the abundance of Akkermansiaceae and is suffering from prostate cancer.

10. Combination according to claim 1 for use in the treatment of a patient that is in need of increasing the abundance of Akkermansia muciniphila in the large intestine.

11. Combination for the use according to claim 10, wherein said patient is suffering from one or more of the following: obesity, diabetes, intestinal inflammation, liver disease, autism, atopy, metabolic disorder, and chronic alcohol consumption.

12. Combination comprising vitamin B2 and Lactobacillus rhamnosus for use in increasing the abundance of Verrucomicrobia, Akkermansiaceae, and/or Akkermansia in the large intestine of an animal, preferably a human, wherein said use comprises delivering the vitamin B2 and the Lactobacillus rhamnosus to the large intestine.

13. Combination comprising vitamin B2 and Lactobacillus rhamnosus for the use according to claim 12, wherein the vitamin B2 and the Lactobacillus rhamnosus are delivered to the large intestine by a delayed-release formulation.

14. Combination comprising vitamin B2 and Lactobacillus rhamnosus for the use according to claim 12, wherein said use comprises administering the vitamin B2 and the Lactobacillus rhamnosus simultaneously and/or sequentially to the animal, preferably a human.

15. Combination comprising vitamin B2 and Lactobacillus rhamnosus for the use according to claim 12, wherein the animal, including a human, is experiencing a condition selected from one or more of the following: prostate cancer, obesity, diabetes, intestinal inflammation, liver disease, autism, atopy, metabolic disorder, and chronic alcohol consumption.
